(19) United States
(12) Patent Application Publication
(10) Pub. No.: US 2025/0283835 A1
(43) Pub. Date: Sep. 11, 2025

(54) INSPECTION DEVICE FOR AUTOMATED WELD INSPECTION, AND METHOD FOR WELD INSPECTION

(71) Applicant: EEW SPECIAL PIPE CONSTRUCTIONS GMBH, Rostock (DE)

(72) Inventor: Matthias SCHWARZ, Neubukow (DE)

(73) Assignee: EEW SPECIAL PIPE CONSTRUCTIONS GMBH, Rostock (DE)

(21) Appl. No.: 19/065,421

(22) Filed: Feb. 27, 2025

(30) Foreign Application Priority Data
Mar. 5, 2024 (EP) 24161584.8

(51) Int. Cl. G01N 21/952; G01N 33/2045

(52) U.S. Cl. CPC G01N 21/952; G01N 33/2045; G01N 2201/0216; G01N 2201/06113

(57) ABSTRACT

A mobile inspection device for inspecting round welds of a large pipe includes at least one movable inspection carriage as a mobile base unit, wherein the inspection carriage has at least three wheels which are mounted on the base unit, at least one measuring unit for inspecting the weld, at least one transmitting and receiving unit, and at least one control unit. The inspection device also includes a marking unit. Also provided is a method for inspecting a round weld on a large pipe.

The invention relates to a mobile inspection device for inspecting round welds of a large pipe according to the generic part of claim 1, and to a method for weld inspection according to claim 14.

An important aspect for the quality of welded pipes made of multiple pipe elements is the quality of the round welds. The welding of the round seams typically occurs using submerged arc welding. Specifically for large pipe having a diameter of more than 6 m which is used in the field of offshore foundations, a high inspection coverage (100%) of all seams is required in order to ensure the fatigue strength over at least 20 years. In accordance with the applicable DIN ISO 5817 standard, a visual inspection by eye shall take place. The standard thereby describes the optically different seam shapes and the imperfections that arise therefrom. If imperfections or defects are found during the visual inspection, they are marked by hand so that a reworking can take place. The imperfections are also noted in an inspection report.

The cost for the weld inspection is therefore very high since sufficient personnel must be used for a complete inspection of all welds. Several problems arise during the visual inspection. The human eye becomes fatigued in the course of the workday, and becomes less accurate during the inspection. The visual inspection always results in a subjective evaluation, since each inspector inspects with a different level of discrimination. Additionally, it is hardly possible to document the visual inspection, since photos only convey a two-dimensional image, but do not depict the three-dimensional nature, and inspection reports only provide an incomplete report of the condition of the seam.

Thus, there are efforts to perform the inspection of welds mechanically. DE 10134696 C1 describes a device for ultrasonic weld testing of longitudinally welded pipes for longitudinal and transversal errors. For the testing, the device comprises two testing carriages swingably suspended and movable on the pipe surface to the right and left of the weld for longitudinal flaw inspection, and one testing carriage swingably suspended in central relationship to the weld and movable on the pipe surface for transverse flaw inspection. The device has as a relatively complicated design and requires multiple testing carriages for the various inspection tasks.

The object of the present invention is to overcome the aforementioned disadvantages and, in particular, to reduce the subjective portion of the evaluation, increase the speed of the inspection, and unburden the people tasked with the inspection.

According to the invention, the object is attained with a mobile inspection device for inspecting round welds of a large pipe according to claim 1. Furthermore, the object is attained with a method for inspecting a round weld on a large pipe according to claim 12.

Further embodiments are the subject matter of the dependent claims or described below.

The mobile inspection device according to the invention for inspecting round welds of a large pipe comprises at least the following components:
a movable inspection carriage as a mobile base unit, wherein the inspection carriage has at least three wheels which are mounted on the base unit;
at least one measuring unit for inspecting the weld;
at least one transmitting and receiving unit;
at least one control unit; and
if necessary, a drive unit which drives the inspection carriage, for example at least one electric motor.

The measuring unit, the transmitting and receiving unit and, if necessary, the drive unit, and where applicable the control unit, are attached to the mobile base unit, that is, to the inspection carriage, and are carried by said base unit. The measuring unit is a laser unit with which the quality of the weld is measured. The quality of the weld comprises the height profile of the weld and the width of the weld. The inspection device comprises at least one marking unit which is attached to the mobile base unit. Using the marking unit, imperfections or sites requiring a reworking or an expert opinion are marked.

Preferably, the measuring unit is a laser unit which has at least one laser head with an emitter and with a detector, wherein the emitter emits at least one laser beam and the detector captures the reflected laser beam. The laser unit preferably comprises a light laser.

The laser head is preferably attached to the base unit via a mounting device with an adjusting device, wherein the distance between the pipe surface and laser is adjusted using the adjusting device. The adjusting device is a set screw or another setting device, for example. With the adjusting device, the distance between the laser unit and pipe surface is adjusted and thus adapted to the inner diameter and outer diameter of the pipe to be inspected.

The inspection carriage can comprise a steering unit, wherein the steering unit is connected to the wheels and the steering unit steers the wheels.

The steering unit can be connected to the control unit, wherein the control unit controls the steering unit and the control unit receives the signals via a manual control, for example via a control using a joystick or control lever, or via an automated control using software.

If the inspection carriage comprises at least one drive unit that drives the inspection carriage, for example at least one electric motor, the drive unit can be connected to at least one control unit. The control unit can control the forward or backward movement of the inspection carriage and the speed thereof.

The inspection device can comprise at least one power supply, preferably a storage battery, a battery, or a power supply line. The energy supply can be embodied as an internal or external storage battery or battery pack and can supply the drive unit, the pump, the control unit, and all electrical or electronic systems of the inspection device with current.

With an automated control of the inspection carriage, the control can occur via software which has a programmed seam detection. The measuring unit detects the location of the seam and transmits the information to the control unit via the transmitting unit. The control unit uses this to determine the relative position of the inspection carriage to the seam. When a predefined threshold value is reached, the control unit transmits a signal to the steering unit and the steering unit corrects the steering to the left or right in order to align the inspection carriage relative to the seam.

It is advantageous if the inspection device has a seam detection and the inspection carriage is controlled based on the data from the seam detection.

A control unit can be embodied as a chip, as a software unit, or as a computing unit, and can be enclosed in an instrument housing with a filter system. A positive pressure is preferably applied to the housing interior.

As a result of the enclosure of the control unit in a separate instrument housing having, if necessary, a filter system on the housing and, where applicable, positive pressure in the housing, the ingress of metal dust or other contaminants is prevented, which would cause contamination of the control unit in the long run. There is a short-circuit hazard for the control unit due to the contaminants, which hazard is avoided with the aid of the housing.

A control unit can be arranged in the inspection carriage or externally, for example in an external container. In addition to a control unit for at least one marking unit, the inspection device can also comprise control units for the inspection carriage, for example for a steering unit and/or a drive unit of the inspection carriage, and a control unit for the measuring unit.

The inspection device can comprise an evaluation unit which evaluates the data obtained from the measuring unit, such as the measured values for example, wherein the evaluation unit preferably comprises a storage unit which stores the data obtained from the measuring unit and the data generated by the evaluation unit.

In one embodiment, the marking unit comprises at least one marking head, for example a print head, at least one reservoir for paint, and a paint line between the marking head and reservoir. Because of the construction of the marking unit on the inspection carriage, the paint line can be kept as short as possible, so that paint only needs to be transported over a short distance. The paint line or paint lines form a paint guiding system with which paint, for example ink, from the reservoir, for example a paint bag, is guided to the marking head.

The marking unit can comprise a holder and, using the holder, can be attached to a mounting device, if necessary with an adjustment device, on the base unit. Via an adjustment device, the distance between the pipe surface and the marking head can adjusted. The adjusting device is a set screw or other setting device, for example. With the adjusting device, the distance from the pipe surface can be adjusted and thus adapted to the inner diameter and outer diameter of the pipe to be inspected.

The marking heads of the marking unit are preferably connected to the control unit, and the control unit controls the marking heads. With a corresponding signal, the marking heads dispense paint and thereby mark a site on the pipe surface or on the seam. The duration and amount of the paint dispensed can be controlled by the signal. The marking heads can be embodied to be stationary or movable. In the case of movable marking heads, the control unit can also control the movement and positioning of the marking heads.

The actuation of the marking heads via the control unit can take place based on a real-time evaluation of the data from the measuring unit. The marking unit can thereby be positioned, for example, with the marking head on the left seam edge, the right seam edge, or the center of the weld. Accordingly, the measuring unit can measure the weld at the left seam edge, the right seam edge or the center of the weld.

The marking unit can comprise a pressure-controlled hose pump which is connected to the reservoir, wherein the hose pump pumps the paint from the reservoir to the marking head. The hose pump preferably comprises a diaphragm pressure switch. The hose pump is preferably operated using 24 V. The marking unit preferably requires 1 bar of working pressure. Since compressed air lines have a high weight, the realization of the positive pressure of 1 bar in the paint line via the pressure-controlled hose pump offers the advantage that the design has a lower weight and therefore adheres more easily to the pipe surface. The diaphragm pressure switch opens and closes a contact in order to maintain the line pressure via the hose pump.

The reservoir of the marking unit can comprise a fixture with a bearing, and can be rotatably mounted on the base unit using the fixture. As a result of the rotatable mounting, the paint fill level is equalized in an overhead situation, so that paint can be delivered.

The wheels are preferably embodied as magnetic wheels.

The base unit preferably comprises at least two sets of wheels, wherein each wheel set has two wheels and the wheels are rotatably mounted on an axle, and wherein one wheel of the wheel set is respectively arranged on the right side of the base unit and wherein one wheel of the wheel set is respectively arranged on the left side of the base unit.

Furthermore, the subject matter of the invention is a method for inspecting a round weld on a pipe. The method according to the invention comprises the steps:
a) providing a pipe having a round weld, if necessary on a turning device;
b) providing an inspection device according to one of patent claims 1 through 11;
c) placing the inspection carriage of the inspection device onto the pipe surface in the region of the weld;
d) moving the inspection carriage of the inspection device along the weld and simultaneously measuring the quality of the weld using the measuring unit by emitting a laser beam with at least one emitter and capturing the reflected beam with a detector;
e) transmitting the measured values (actual values) to a control unit and comparing a measured value with a target value stored on the control unit;
f) if a measured value deviates from the target value, transmitting a signal to the marking unit, activating the marking unit, and marking the pipe surface using the marking unit.

In a further step, the measured values transmitted in step e) can be transmitted to a storage unit and stored on the storage unit.

In step a), the pipe can be provided on a turning device. In this case, the pipe is turned about its longitudinal axis against the direction of travel of the inspection carriage using the turning device while the inspection carriage moves along the weld according to step d).

The pipe has a diameter of at least 6 m, preferably a diameter of at least 8 m or 10 m.

The method for examining a round weld of a large pipe thus enables an automated inspection of the weld. The inspection carriage with the wheels is placed onto the pipe, wherein the wheels are preferably magnetic so that the inspection carriage adheres to the pipe surface. The inspection carriage preferably carries a line laser as a measuring unit. The laser scans the height profile of the weld and measures the width of the weld. The laser unit and the inspection carriage are coupled to a control unit, that is, to a measuring station. The marking unit mounted on the inspection carriage marks the defective sites of the weld.

The mobile inspection device according to the invention and the inspection method according to the invention have a host of advantages over the conventional visual inspection.

Large pipes, such as those used for offshore foundations, have a pipe diameter of greater than 6 m, wherein pipe diameters of 8 m or 10 m are not uncommon. Even pipe diameters of 11.50 m or 12 m are possible, for example. The round weld of a large pipe of this type therefore has a considerable length, both on the inside of the pipe but especially on the outside of the pipe.

The pipes to be inspected are longitudinal-seam-welded and round-seam-welded large pipes. Longitudinal-seam-welded large-pipe elements are manufactured from steel sheet panels. The cut sheet panel is first bent into a round shape, and the open edges of the resulting open-seam pipe element are tacked together using a temporary seam (tack welding) that is removed again later in the production process. The tack-welded pipe element is positioned in a welding bay, and the inner seam is made on the pipe element first and then the outer seam. The pipe can then be further processed, for example coated. In a typical construction, the pipes have a wall thickness of 35 mm to 250 mm, preferably 40 to 200 mm, particularly preferably 50 mm to 170 mm. Large pipes and large-pipe elements with a diameter of 6 m to 10 m preferably have a wall thickness of 50 mm to 170 mm. A large-pipe element typically has a length of 2 m to 4.2 m. The pipes are welded together from a plurality of pipe elements and thereby have lengths of up to 120 m. The pipe elements are connected to one another by submerged arc welding, wherein the welds that are to be inspected are created. In these dimensions, the pipes weigh on average over several thousand tons.

With the inspection device, a very high inspection speed is possible. The inspection carriage moves over the pipe at a speed of 70 mm/s, for example. In the case of a pipe having a pipe diameter of 8 m, this results in an inspection time of only 6 minutes. With the visual inspection, 20 minutes have been necessary until now. In the case of a pipe diameter of 10 m, an inner round seam can be inspected in approximately 8 min using the inspection device. By comparison, the visual inspection of the inner seam takes at least 30 min.

Additionally, the necessary movement of the pipe is reduced. For seam inspection, in particular the outer seam inspection, the pipe is typically mounted on a turning device and turned about its longitudinal axis. Whereas a complete rotation of the pipe is necessary to inspect the outer round weld and the inner pipe seam for the visual inspection, no rotation at all, or merely a half rotation, of the pipe is required with the inspection device. The pipe is turned against the direction of travel of the robot when the outer seam is measured; less turning is required as a result. For an inspection of the outer seam in the case of a pipe in relative motion, that is, when the inspection carriage moves along the weld and the pipe is turned in the opposite direction, the inspection duration of a pipe having a 10 m diameter is approximately 15 min. By comparison, the visual inspection of the outer seam takes at least 30 min.

The speed of the inspection is thus significantly increased compared with the visual inspection. The human influence during the weld inspection and the subjective evaluation are eliminated by the use of the inspection device and the method. The employees are unburdened, since the task is very tiring.

If a real-time evaluation takes place via software, this has the advantage that the marking unit can be immediately actuated and a tagging of irregularities can take place directly on the pipe surface.

If the inspection device according to the invention is equipped with a storage unit or connected to a storage unit, the data measured by the measuring unit can be stored as an image or height file. An advantage is that all data are recorded, including the marked switching points, and that a documentation of the quality and of the inspection of the weld is thus created. After the sites marked as defective have been worked on, a new measurement takes place in order to document the remediation.

The data recording offers a documentation from which the exact condition of the weld follows when the pipe is delivered for further use.

In a further embodiment, the inspection device comprises an eddy current testing unit in addition to the laser unit. The eddy current testing unit enables a surface crack inspection. For this purpose, the inspection carriage moves over the seams in a second run after measurement of the weld with the laser unit and performs the eddy current test. As a result, the speed of the overall inspection can be significantly increased. In this case, the results of the surface crack inspection can also be recorded and documented.

Additional features, details, and advantages of the invention follow from the text of the claims and from the following description of exemplary embodiments with the aid of the drawings. In the following:

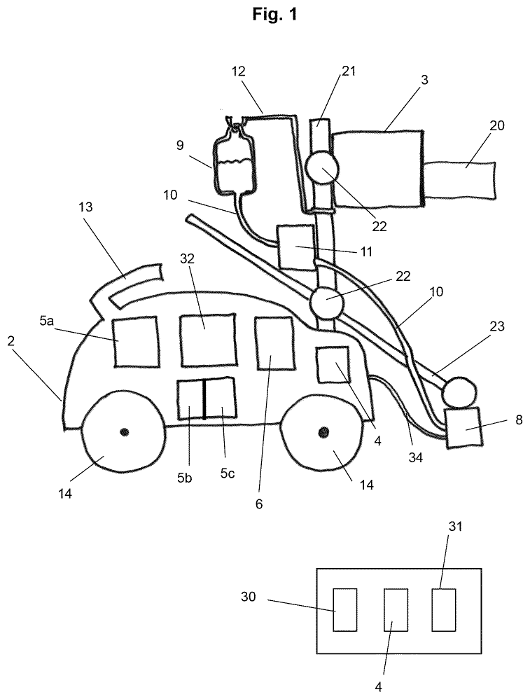

FIG. 1 shows a schematic side view of an embodiment of a mobile inspection device according to the invention,

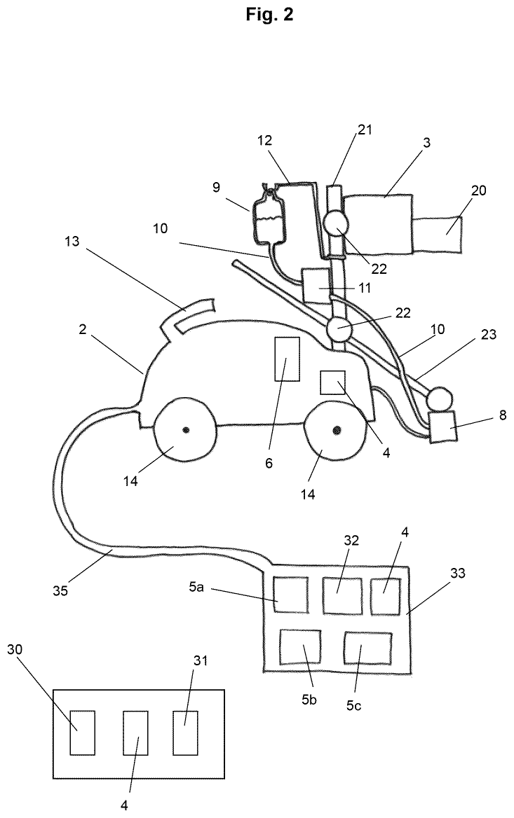

FIG. 2 shows a schematic side view of a further embodiment of a mobile inspection device according to the invention,

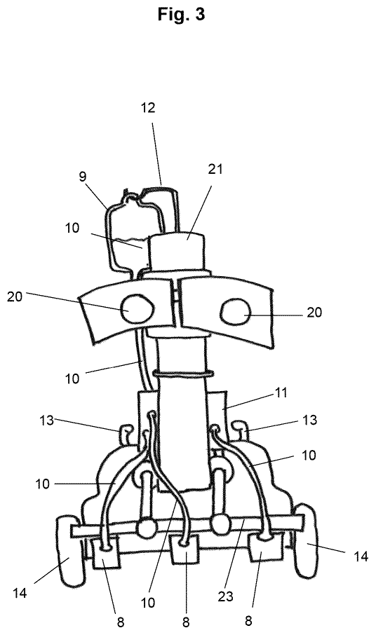

FIG. 3 shows a front view of an inspection device according to the invention,

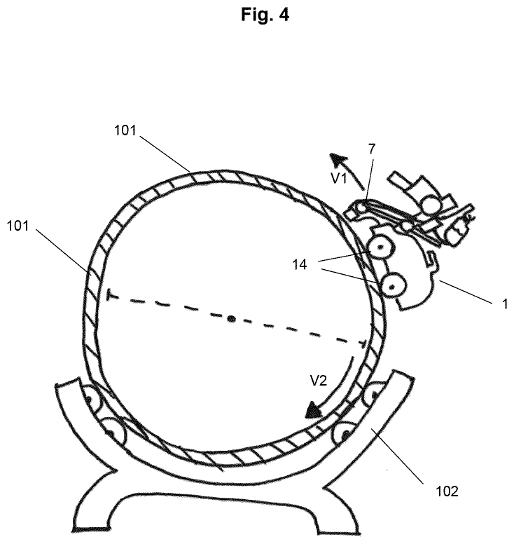

FIG. 4 shows a mobile inspection device during measurement on a large pipe, and

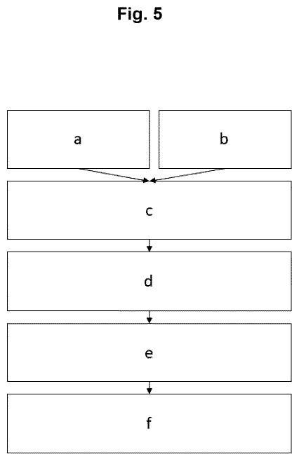

FIG. 5 shows a schematic sequence of a method according to the invention.

FIG. 1 shows a mobile inspection device in a schematic side view. The inspection device 1 for inspecting a round weld of a large pipe comprises a movable inspection carriage 2 as a mobile base unit. In this embodiment, the inspection carriage has four wheels 14 which are respectively mounted in pairs on the base unit 2. In the housing of the inspection carriage, a transmitting and receiving unit 4, a control unit 5a for the inspection carriage, a control unit 5b for the marking unit, a control unit 5c for a measuring unit, a drive unit 6, and a storage battery 32 are arranged. The drive unit 6 drives a pair of the wheels 14. On one side of the inspection carriage 2, a measuring unit 3 for inspecting the weld can be seen. The measuring unit 3 comprises a laser unit having a laser head 20 with an emitter. The emitter can emit at least one laser beam. Adjacent to the laser head, a detector is arranged (not shown) which captures the reflected laser beam. The measuring unit 3 is mounted on the inspection carriage using a mounting device 21. An adjusting device 22 is located on the mounting device 21, with which adjusting device 22 the position of the measuring unit can be adjusted in order to obtain an optimal measuring result.

On the same side on the inspection carriage as the measuring unit, a marking unit 7 is mounted which comprises multiple marking heads 8, of which only one can be seen. The marking heads 8 are connected to a reservoir 9 for paint via a paint line 10. Between the marking heads 8 and the reservoir 9, a pressure-controlled hose pump 11 is positioned which pumps paint from the reservoir 9 to the marking head 8. The reservoir 9 is attached to the mounting device 21 via a fixture 12. The marking head 8 is connected via a control line 34 to the control unit 5b in the interior of the inspection carriage. The marking heads are mounted on the mounting device 21 via a holder 23. The position thereof can be adjusted via an adjusting device 22.

On the inspection carriage, a handle 13 is also affixed with which the inspection carriage can be lifted and transported. Additionally, an evaluation unit 30 and a storage unit 31 are externally provided which evaluate the measured data and store measured data or evaluation results, respectively.

FIG. 2 shows a further embodiment of the inspection device 1 according to the invention. In this embodiment, multiple control units 5 are arranged not in the inspection carriage, but rather externally, for example in an external control module 33. The external control module 33 contains a transmitting and receiving unit 4, a control unit 5a for the inspection carriage, a control unit 5b for the marking unit, a control unit 5c for a measuring unit, and a storage battery 32. Power and signals are transported between the inspection carriage and control unit via a supply line 35. Additionally, an evaluation unit 30 and a storage unit 31 are also externally provided in this case, which evaluation unit 30 and storage unit 31 evaluate the measured data and store measured data or evaluation results, respectively.

FIG. 3 shows a schematic front view of an inspection device. The measuring unit 3 is mounted on the inspection carriage 2 via a mounting device 21. The measuring unit comprises two adjacently arranged laser heads 20. The marking unit 7 has three adjacently arranged marking heads 8. The marking heads 8 are respectively connected to a pump 11 via a paint line 10, which pump 11 is connected to a reservoir 9 for paint via a further central paint line. In the measuring unit 3, two laser heads 20 can be seen which are arranged adjacently.

In FIG. 4, a mobile inspection device 1 can be seen during measurement on a large pipe 100. The wheels 14 of the inspection carriage rest on the pipe surface 101. The marking unit 7 is arranged a short distance above the pipe surface. The inspection carriage moves forward in the direction of the arrow V1. The large pipe 100 rests on a turning device 102 which can turn the large pipe in the direction of the arrow V2.

FIG. 5 shows a schematic sequence of a method according to the invention for inspecting a round weld on a large pipe. In step a), a pipe having a round weld is provided, if necessary on a turning device. At the same time, an inspection device according to the invention is provided in step b). In step c), the inspection device is placed onto the pipe surface in the region of the weld. In the step d) that follows, the inspection carriage of the inspection device moves along the weld and simultaneously measures the quality of the weld using the measuring unit by emitting a laser beam with at least one emitter and capturing the reflected beam with a detector. According to step e), the measured values (actual values) obtained are transmitted to a control unit and a measured value is compared with a target value. If a deviation of a measured value from the target value is found, in step f) a signal is transmitted to a marking unit, the marking unit is activated, and the pipe surface is marked by the marking unit.

The invention is not limited to any one of the embodiments described above, but rather can be modified in many and varied ways.

All features and advantages arising from the claims, the specification, and the drawing, including constructive details, spatial arrangements, and method steps, can be essential to the invention both separately and also in the most widely different combinations.

List of Reference Symbols

| | |
|---|---|
| Mobile inspection device | 1 |
| Inspection carriage/base unit | 2 |
| Measuring unit | 3 |
| Transmitting and receiving unit | 4 |
| Control unit | 5 |
| Drive unit | 6 |
| Marking unit | 7 |
| Marking head | 8 |
| Reservoir for paint | 9 |
| Paint line | 10 |
| Hose pump | 11 |
| Fixture | 12 |
| Handle | 13 |
| Wheel | 14 |
| Laser head | 20 |
| Mounting device | 21 |
| Adjusting device | 22 |
| Holder | 23 |
| Evaluation unit | 30 |
| Storage unit | 31 |
| Storage battery | 32 |
| Control module | 33 |
| Control line | 34 |
| Supply line | 35 |
| Large pipe | 100 |
| Pipe surface | 101 |
| Turning device | 102 |
| Arrow for movement direction of inspection carriage | V1 |
| Arrow for movement direction of pipe | V2 |

1. A mobile inspection device for inspecting a round weld of a large pipe, comprising:
a movable inspection carriage as a mobile base unit, comprising at least three wheels-which are mounted on the base unit,
at least one marking unit which is attached to the mobile base unit,
at least one measuring unit for inspecting the weld,
at least one transmitting and receiving unit, and
at least one control unit, wherein
the at least one measuring unit, the at least one transmitting and receiving unit, and the at least one control unit are attached to the mobile base unit, and the measuring unit is a laser unit.

2. The mobile inspection device according to claim 1, wherein the laser unit has at least one laser head with an emitter which emits at least one laser beam, and which has at least one detector which captures the reflected laser beam, wherein the laser unit is a light-laser unit.

3. The mobile inspection device according to claim 2, wherein the measuring unit is attached to the base unit via a mounting device with an adjusting device and the distance between the pipe surface and laser is adjusted using the adjusting device.

4. The mobile inspection device according to claim 1, wherein the inspection carriage comprises a steering unit, the steering unit is connected to the wheels, and the steering unit steers the wheels.

5. The mobile inspection device according to claim 1, wherein the at least one control unit is a chip or a software unit and the at least one control unit is enclosed in an instrument housing with a filter system, and a positive pressure is applied to the housing interior.

6. The mobile inspection device according to claim 1, wherein the at least one marking unit comprises at least one marking head, at least one reservoir for paint, and a paint line between the at least one marking head and the at least one reservoir.

7. The mobile inspection device according to claim 6, wherein the at least one marking head of the at least one marking unit is connected to the at least one control unit and the at least one control unit controls the at least one marking head.

8. The mobile inspection device according to claim 6, wherein the at least one marking unit comprises a pressure-controlled hose pump, the pressure-controlled hose pump is connected to the at least one reservoir, and the pressure-controlled hose pump pumps the paint from the at least one reservoir to the at least one marking head wherein the pressure-controlled hose pump comprises a diaphragm pressure switch.

9. The mobile inspection device according to claim 6, wherein the at least one reservoir comprises a fixture with a bearing and is rotatably mounted on the base unit with the fixture.

10. The mobile inspection device according to claim 1, wherein the at least three wheels are magnetic wheels.

11. The mobile inspection device according to claim 1 further comprises; an evaluation unit-which evaluates the data obtained from the at least one measuring unit and the evaluation unit preferably comprises a storage unit which stores the data obtained from the at least one measuring unit and the data generated by the evaluation unit.

12. A method for inspecting a round weld on a large pipe, comprising:
providing a pipe having a round weld on a turning device;
providing the mobile inspection device according to claim 1;
placing the mobile inspection device onto a pipe surface in a region of the weld;
moving the movable inspection carriage of the mobile inspection device along the weld and simultaneously measuring the quality of the weld using the at least one measuring unit by emitting a laser beam with at least one emitter and capturing the reflected beam with a detector;
transmitting the measured values to the at least one control unit and comparing a measured value with a target value; and
if a measured value deviates from the target value, transmitting a signal to the at least one marking unit, activating the at least one marking unit, and marking the pipe surface using the at least one marking unit.

13. The method for inspecting the round weld on the large pipe according to claim 12, wherein the measured values transmitted in e) are transmitted to a storage unit and are stored on the storage unit.

14. The method for inspecting the round weld on the large pipe according to claim 12, wherein in a), the pipe is provided on the turning device and that, while the movable inspection carriage moves along the weld according to d), the pipe is turned about its longitudinal axis against a direction of travel of the movable inspection carriage.

15. The method according to claim 12, wherein the pipe has a diameter of at least 6 m.

16. The mobile inspection device according to claim 1 further comprising:
a drive unit.
